(19) United States
(12) Patent Application Publication
(10) Pub. No.: US 2024/0100110 A1
(43) Pub. Date: Mar. 28, 2024

(54) PAIN RELIEF SALVE AND A METHOD FOR MAKING SUCH

(71) Applicants: Jesus Arellano, Canton, GA (US); Jason Arellano, Canton, GA (US); Samuel Hardin, Canton, GA (US)

(72) Inventors: Jesus Arellano, Canton, GA (US); Jason Arellano, Canton, GA (US); Samuel Hardin, Canton, GA (US)

(21) Appl. No.: 17/934,626

(22) Filed: Sep. 23, 2022

(51) Int. Cl. A61K 36/185; A61K 9/06; A61K 31/05; A61K 47/44

(52) U.S. Cl. CPC A61K 36/185; A61K 9/06; A61K 31/05; A61K 47/44; A61K 2236/15; A61K 2236/17

(57) ABSTRACT

A method for producing composition for a pain-relieving salve by producing a decarbonated cannabinoid and placing the decarbonated cannabinoid and a predetermined amount of oil into a sealed container, placing the sealed container in a pressure cooker with a predetermined volume of liquid, pressure cooking the sealed container at a predetermined temperature for a predetermined time, cooling the sealed container to room temperature, and separating an infused oil of the sealed container by straining contents of the sealed container.

### BACKGROUND OF THE INVENTION

The present invention relates to a salve and, more particularly, to a pain relief salve and a method for making such.

Currently, most pain relief salves on the market provide a methanol formula for aching muscles and joints. These products may irritate a user's skin and often contain unnatural ingredients. Many users are sensitive to these products and their burning sensation. Many users simply prefer natural ingredients.

As can be seen, there is a need for a pain relief salve comprised of natural ingredients and does not include methanol or a similar ingredient.

### SUMMARY OF THE INVENTION

In one aspect of the present invention, a method for producing composition for a pain-relieving salve comprises producing a decarbonated cannabinoid by a method comprising grinding a cannabinoid flower to form a ground cannabinoid flower, baking the ground cannabinoid flower at a predetermined temperature and for a predetermined time, and cooling the ground cannabinoid flower to room temperature. Then placing the decarbonated cannabinoid and a predetermined amount of oil into a sealed container, placing the sealed container in a pressure cooker with a predetermined volume of liquid, pressure cooking the sealed container at a predetermined temperature for a predetermined time, cooling the sealed container to room temperature, and separating an infused oil of the sealed container by straining contents of the sealed container.

These and other features, aspects and advantages of the present invention will become better understood with reference to the following drawings, description, and claims.

### BRIEF DESCRIPTION OF THE DRAWINGS

FIG. 1 is a flow diagram detailing method steps for a production of a decarbonated cannabinoid according to an embodiment of the present invention;

FIG. 2 is a flow diagram detailing method steps for production of an infused coconut oil with the decarbonated cannabinoid according to an embodiment of the present invention;

FIG. 3 is a flow diagram detailing method steps for production of an infused olive oil with the decarbonated cannabinoid according to an embodiment of the present invention;

FIG. 4 is a flow diagram detailing method steps for straining the infused coconut oil according to an embodiment of the present invention;

FIG. 5 is a flow diagram detailing method steps for straining the infused olive oil according to an embodiment of the present invention;

FIG. 6 is a flow diagram detailing method steps for production of a lavender salve according to an embodiment of the present invention;

FIG. 7 is a flow diagram detailing method steps for production of a lemon, lavender salve according to an embodiment of the present invention; and

FIG. 8 is a flow diagram detailing method steps for production of a eucalyptus salve according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims with reference to the drawings.

A general overview of the various features of the invention will be provided, with a detailed description following. Broadly, an embodiment of the present invention provides a pain-relieving salve, which may be a lotion or a rub composed of natural ingredients. The salve may be utilized for but is not limited to minor aches, pain, sun burn, dry skin, arthritis, and carpel tunnel among other things. The salve may be applied topically by rubbing onto a user's skin.

The salve of the present invention may comprise a cannabinoid such as or comprising primarily cannabigerol (CBG) or cannabidiol (CBD). CBG and CBD may be utilized interchangeably herein. The salve may further comprise an oil and/or essential oils such as yucca root, lavender, spearmint, and/or deep blue.

Olive oil may be utilized to promote faster, easier absorption of the present invention into the user's skin. Similarly, coconut oil may be utilized.

Yucca root may oil increase a potency of the present invention as well as relieve a user's headaches.

A combination of yucca root oil and a cannabinoid in a salve promotes pain relief and therapy for users.

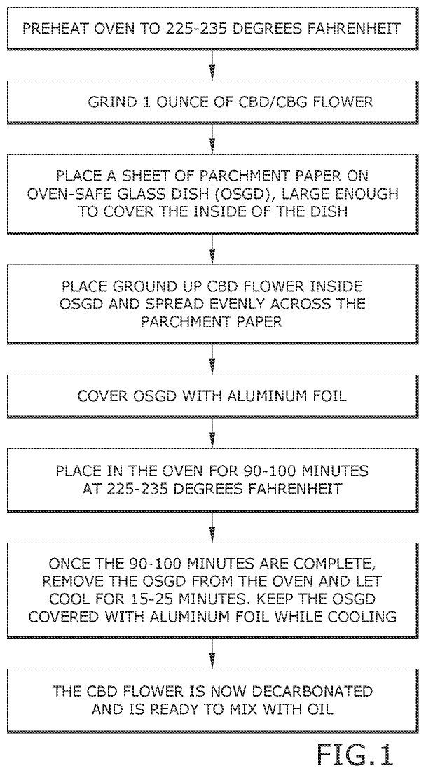

The present invention may be formed by the method steps described in the Figures. The temperatures, weights, and times detailed in the Figures are by way of example. The temperatures, weights, and times may be predetermined. They are approximations based on experimentation and may be scaled in relationship to each other. The scaling may be performed proportionally to a weight of an ingredient such as a decarbonated cannabinoid wherein a decarbonated cannabinoid is a cannabinoid baked adequately and sufficiently as described in FIG. 1.

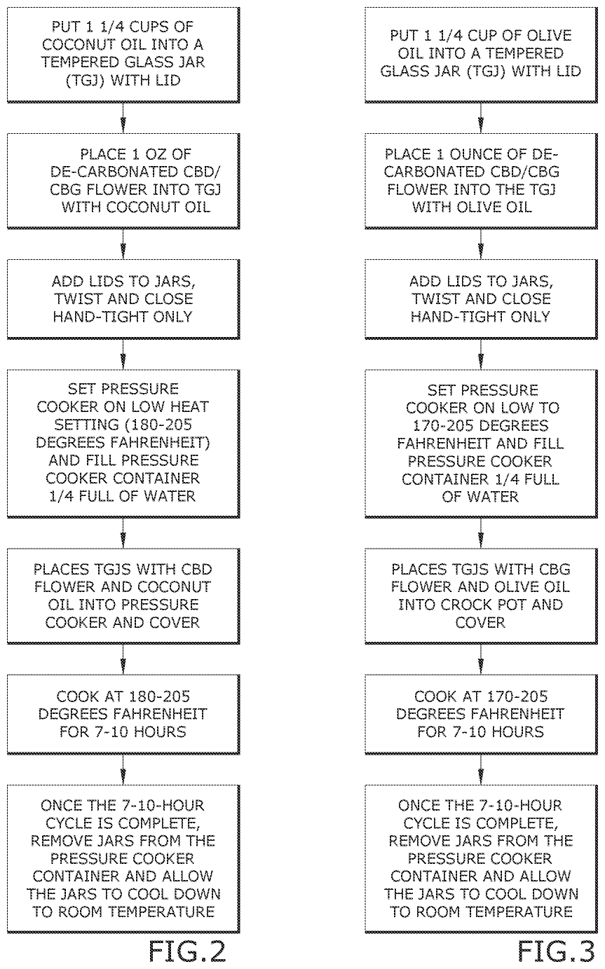

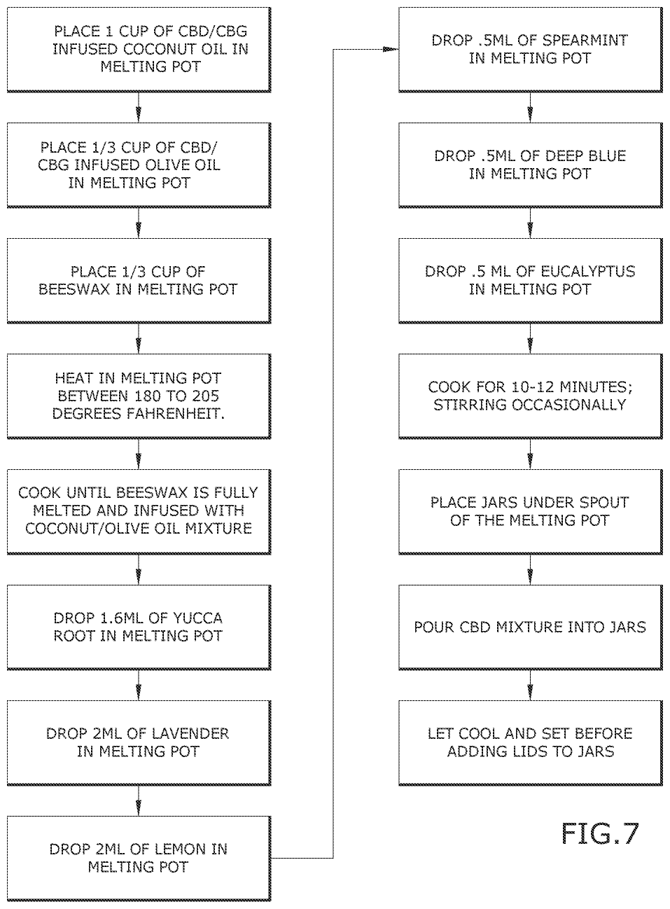

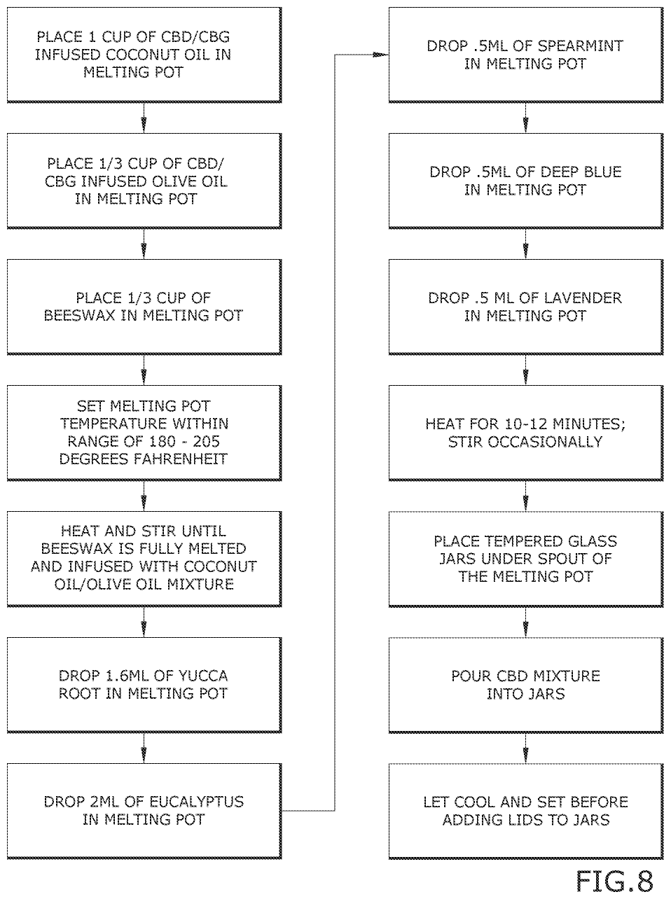

Referring now to FIGS. 1-8, FIG. 1 details a de-carbonation process. The decarbonation process may be utilized for cannabinoids such as cannabigerol (CBG) and cannabidiol (CBD). A cannabinoid flower may be ground and placed on a parchment paper covering a dish. The covering may shield it, reflect heat, and encapsulate moisture during a baking/drying process. The ground cannabinoid may be covered with aluminum foil and baked at approximately 225-325 degrees Fahrenheit for approximately 90-100 minutes. A lower temperature or a shorter time period may result in cannabinoid with an excess of carbonation sufficient for an infusion process. A higher temperature or longer time period may result in a burnt cannabinoid. The cannabinoid may be removed from the oven remain covered for an additional 15-20 minutes to cool. The baked, decarbonated cannabinoid may be infused with oils as described in FIGS. 2 and 3.

FIGS. 2 and 3 detail an infusion of the baked cannabinoid with a coconut oil and an olive oil, respectively, to form an infused oil. A quantity of oil, such as approximately 1.25 cups for approximately 1 ounce (oz) of baked cannabinoid, may be placed in a tempered glass jar and mixed with the baked cannabinoid. The jar may be sealed and placed in a pressure cooker. A predetermined quantity or volume of liquid may be added to the pressure cooker. For example, a volume of the pressure cooker may be filled approximately a quarter full with water. The pressure cooker may then be operated or powered on for approximately 7-10 hours and at a temperature of approximately 180-205 degrees Fahrenheit for the coconut oil infusion or 170-205 degrees Fahrenheit for the olive oil infusion. During the 7-10 hours, the temperature is made steady. At said time and temperature, a maximum amount of oil is absorbed. Going above that time or temperature may lead to degradation in the of the product leading to a less potent product. In some embodiments, coconut oil may be pressure cooked for 7 hours at approximately 180 degrees Fahrenheit. In some embodiments, coconut oil or olive oil may be pressure cooked for 10 hours at approximately 205 degrees Fahrenheit. In some embodiments, olive oil may be pressure cooked for 7 hours at approximately 170 degrees Fahrenheit. The jars may then be removed from the pressure cooker and cooled to room temperature.

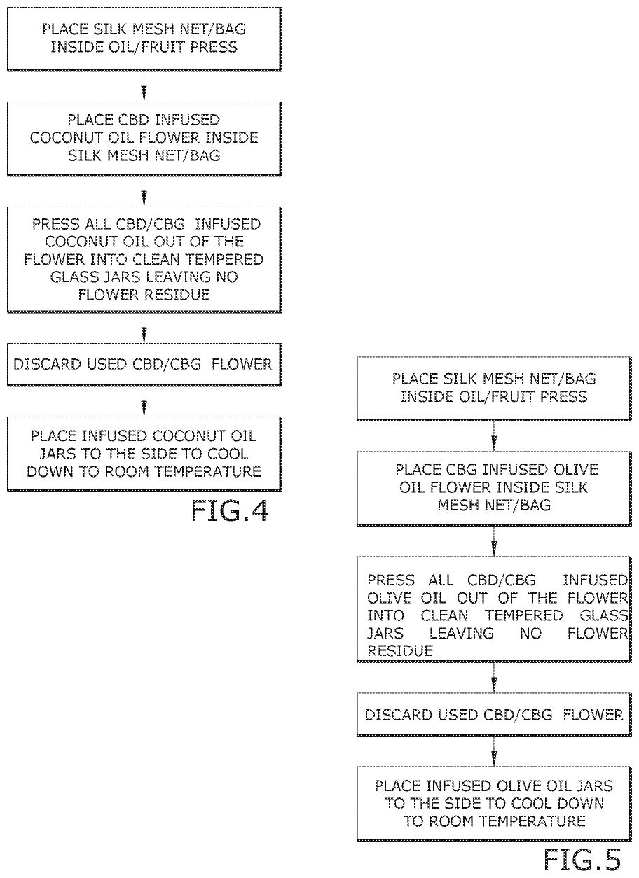

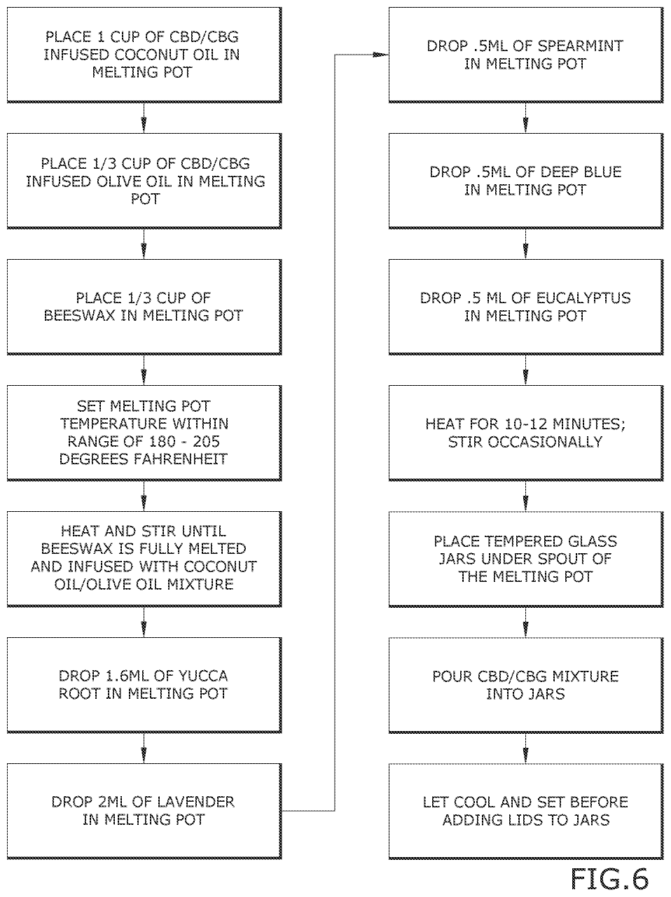

FIGS. 4 and 5 detail straining the infused coconut oil and the infused olive oil, respectively. The infused oil may be placed into a mesh net or bag or fruit press and pressed to strain and remove the flower residue from the infused oil, forming a strained infused oil. The flower residue may be discarded, and the infused oil may be set aside for use as described in FIGS. 6-8. Alternatively, the strained infused oil may be utilized for pain relief and applied to a user.

FIGS. 6-8 detail production of a salve from the strained infused oils. A quantity of the strained infused coconut oil and a quantity of the strained infused olive oil may be added to a receptacle, such as a melting pot, with beeswax. In some embodiments of the present invention, a cup of strained infused coconut oil, a third of a cup of strained infused olive oil, and a third of a cup of beeswax may be used. The receptacle may be heated to a temperature of approximately 180-205 degrees Fahrenheit. A predetermined quantity of essential oils, such as approximately 1.6 milliliters (ml) of yucca root oil, 2 ml of lavender oil, 0.5 ml of spearmint oil, 0.5 ml of deep blue oil, and 0.5 ml of eucalyptus oil may be added to the receptacle. In some embodiments, a predetermined amount of lemon or lemon juice may be added, such as 2 ml. The predetermined quantity of essential oils and/or lemon juice may be scaled at a ratio proportional to the infused oil. The receptacle may be heated to a temperature ranging between 145-155 degrees Fahrenheit and stirred occasionally for approximately 10-12 minutes until the mixture is homogeneous. At 170 degrees Fahrenheit, the beeswax may be burned and that temperature should be avoided. The mixture may then be poured into a container such as a tempered glass jar. The resulting product may be a salve suitable for natural pain relief.

The items listed above and in the Figures, including but not limited to the parchment paper, aluminum foil, the tempered glass jar, oven-safe glass dish, strainer, press, and melting pot amongst others are discussed by way of example only. Any equivalent item may be utilized suitable for achieving a similar purpose or function.

It should be understood, of course, that the foregoing relates to exemplary embodiments of the invention and that modifications may be made without departing from the spirit and scope of the invention as set forth in the following claims.

What is claimed is:

1. A method for producing composition for a pain-relieving salve comprising:
producing a decarbonated cannabinoid by a method comprising:
grinding a cannabinoid flower to form a ground cannabinoid flower;
baking the ground cannabinoid flower at a predetermined temperature and for a predetermined time; and
cooling the ground cannabinoid flower to room temperature;
placing the decarbonated cannabinoid and a predetermined amount of oil into a sealed container;
placing the sealed container in a pressure cooker with a predetermined volume of liquid;
pressure cooking the sealed container at a predetermined temperature for a predetermined time;
cooling the sealed container to room temperature; and
separating an infused oil of the sealed container by straining contents of the sealed container.

2. The method of claim 1, wherein the ground cannabinoid flower is baked at a temperature ranging between 225-235 degrees Fahrenheit and for a time of approximately 90-100 minutes.

3. The method of claim 1, wherein the sealed container is placed in a pressure cooker with the pressure cooker's volume being filled a quarter with water and wherein the sealed container and the water are pressure cooked at a temperature ranging from 170-205 degrees Fahrenheit for a time of 7-10 hours.

4. The method of claim 1, wherein the cannabinoid flower comprises cannabigerol (CBG).

5. The method of claim 1, wherein the cannabinoid flower comprises cannabidiol (CBD).

6. The method of claim 1, wherein the predetermined amount of oil comprises coconut oil and is provided at a ratio of approximately 1.25 cups of oil to 1 ounce of decarbonated cannabinoid.

7. The method of claim 6, wherein the sealed container is placed in a pressure cooker with the pressure cooker's volume being filled a quarter with water and wherein the sealed container and the water are pressure cooked at a temperature ranging from 180-205 degrees Fahrenheit for a time of 7-10 hours.

8. The method of claim 1, wherein the predetermined amount of oil comprises olive oil and is provided at a ratio of approximately 1.25 cups of oil to 1 ounce of decarbonated cannabinoid.

9. The method of claim 1, further comprising:
melting a predetermined amount of infused oil with a predetermined amount of beeswax in a receptacle at a predetermined temperature;
adding a predetermined amount of yucca root oil, a predetermined amount of lavender oil, a predetermined amount of spearmint oil, a predetermined amount of deep blue oil, and a predetermined amount of eucalyptus oil;
applying heat to the receptacle for approximately 10-12 minutes at a temperature of approximately 145-155 degrees Fahrenheit; and
pouring contents of the receptacle into a container and cooling.

10. The method of claim 9, wherein the infused oil and beeswax are melted at a temperature of approximately 180-205 degrees Fahrenheit.

11. The method of claim 9, further comprising adding a predetermined amount of lemon juice.

12. The method of claim 9, wherein the predetermined amount of beeswax is provided at a ratio of ⅓ cup to 1 cup of infused 1⅓ cup of infused oil.
